(19) United States
(12) Patent Application Publication
(10) Pub. No.: US 2018/0078152 A1
(43) Pub. Date: Mar. 22, 2018

(54) AUTOMATED VEHICLE OPERATING SYSTEM WITH DRIVING-RULE MODIFICATION BASED ON OBSERVATION OF ANOTHER VEHICLE

(71) Applicant: Delphi Technologies, Inc., Troy, MI (US)

(72) Inventors: Junqing Wei, Bridgeville, PA (US); Wenda Xu, Pittsburgh, PA (US); Jarrod M. Snider, Pittsburgh, PA (US); Jong Ho Lee, Pittsburgh, PA (US)

(21) Appl. No.: 15/824,037

(22) Filed: Nov. 28, 2017

Related U.S. Application Data
(62) Division of application No. 15/146,354, filed on May 4, 2016.

(51) Int. Cl. A61B 5/026; A61B 5/00; A61B 5/021

(52) U.S. Cl. CPC A61B 5/0261; A61B 5/7278; A61B 5/02125; A61B 2562/04; A61B 5/681; A61B 2562/0238; A61B 2090/306

(57) ABSTRACT

A driving-rule system suitable to operate an automated includes a vehicle-detector and a controller. The vehicle-detector is suitable for use on a host-vehicle. The vehicle-detector is used to detect movement of an other-vehicle proximate to the host-vehicle. The controller is in communication with the vehicle-detector. The controller is configured to operate the host-vehicle in accordance with a driving-rule, detect an observed-deviation of the driving-rule by the other-vehicle, and modify the driving-rule based on the observed-deviation.

### CROSS-REFERENCE TO RELATED APPLICATION

This application claims the benefit under 35 U.S.C. § 120 of U.S. patent application Ser. No. 15/146,534, filed May 4, 2017, the entire disclosure of which is hereby incorporated herein by reference.

### TECHNICAL FIELD OF INVENTION

This disclosure generally relates to a driving-rule system suitable to operate an automated vehicle, and more particularly relates to a system that operates a host-vehicle in accordance with a driving-rule, detects an observed-deviation of the driving-rule by another-vehicle, and modifies the driving-rule based on the observed-deviation.

### BACKGROUND OF INVENTION

Automated or autonomous vehicle are generally programmed to operate in accordance with, i.e. observe, traffic-laws. Automated vehicles may also be programmed to operate in accordance with driving-rules or guidelines so the automated vehicles behave in a predictable manner. However, in some circumstances strict adherence to these driving-rules may inhibit the general flow of traffic.

### SUMMARY OF THE INVENTION

In accordance with one embodiment, a driving-rule system suitable to operate an automated vehicle is provided. The system includes a vehicle-detector and a controller. The vehicle-detector is suitable for use on a host-vehicle. The vehicle-detector is used to detect movement of an other-vehicle proximate to the host-vehicle. The controller is in communication with the vehicle-detector. The controller is configured to operate the host-vehicle in accordance with a driving-rule, detect an observed-deviation of the driving-rule by the other-vehicle, and modify the driving-rule based on the observed-deviation.

Further features and advantages will appear more clearly on a reading of the following detailed description of the preferred embodiment, which is given by way of non-limiting example only and with reference to the accompanying drawings.

### BRIEF DESCRIPTION OF DRAWINGS

The present invention will now be described, by way of example with reference to the accompanying drawings, in which:

FIG. 1 is a diagram of a driving-rule system in accordance with one embodiment;

FIG. 2 is a traffic scenario encountered by the system of FIG. 1 in accordance with one embodiment; and

FIG. 3 is a traffic scenario encountered by the system of FIG. 1 in accordance with one embodiment.

### DETAILED DESCRIPTION

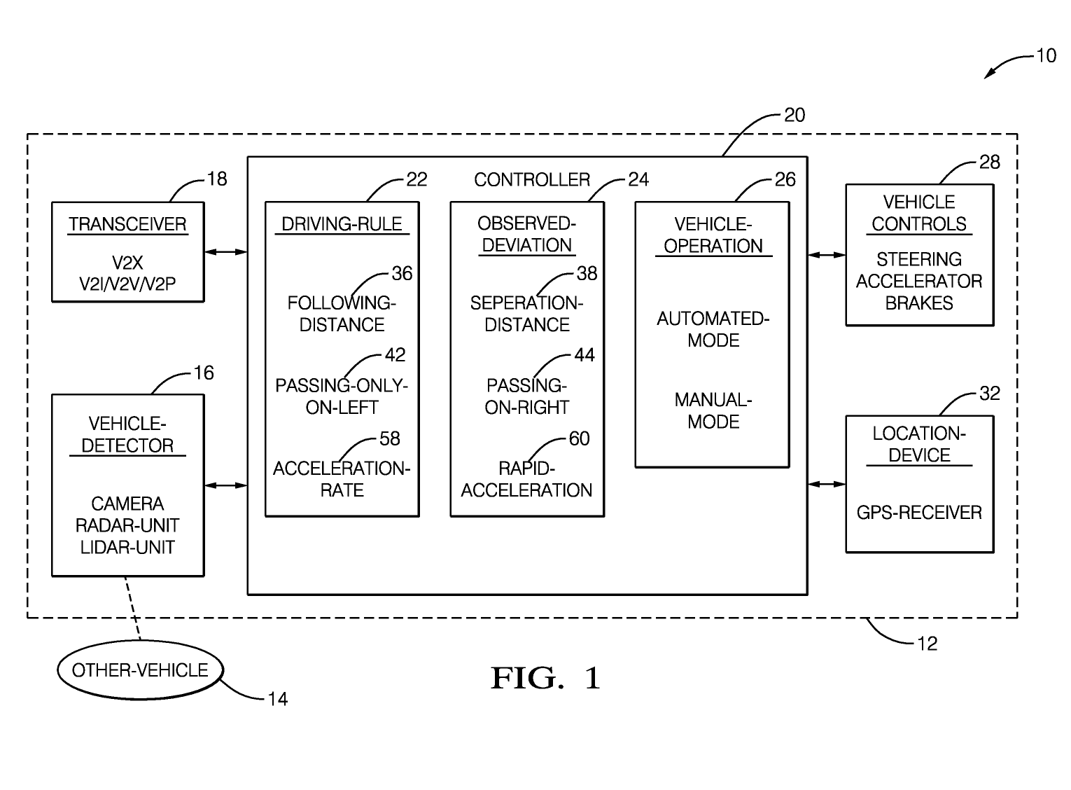

FIG. 1 illustrates a non-limiting example of a driving-rule system 10, hereafter referred to as the system 10. The system 10 is generally suitable to operate an automated vehicle or autonomous vehicle, hereafter referred to as the host-vehicle 12. While the non-limiting examples presented herein are generally related to fully-automated vehicles, i.e. autonomous-vehicles, it is contemplated that the teachings presented herein are also applicable to vehicles that are less than fully-automated, i.e. partially-automated, where an operator (not shown) may manually control some aspect of the host-vehicle 12 such the steering, but the operation of the accelerator and brakes is automated to maintain a predetermined distance behind an other-vehicle 14 forward of the host-vehicle 12.

The system 10 includes a vehicle-detector 16 suitable for use on the host-vehicle 12. By way of example and not limitation, the vehicle-detector 16 may include any one or combination of a camera, a radar-unit, a lidar-unit, an ultrasonic-transducer, or any other sensor technology useful to detect the other-vehicle 14 or objects proximate to the host-vehicle 12. The vehicle-detector 16 is generally used to detect movement of the other-vehicle 14 relative to the host-vehicle 12 and/or any other objects or other vehicles proximate to the host-vehicle 12. The function of the vehicle-detector 16 may also be provided or supplemented by a transceiver 18 configured for vehicle-to-infrastructure (V2I) communications, vehicle-to-vehicle (V2V) communications, and/or vehicle-to-pedestrian (V2P) communications, which may be generically labeled as V2X communications.

The system 10 also includes a controller 20 in communication with the vehicle-detector 16. Signals sent from the vehicle-detector 16 and/or the transceiver 18 may be used to determine the relative speed and/or direction of travel of the other-vehicle 14. The controller 20 is generally configured to operate the host-vehicle 12 in accordance with a driving-rule 22. As used herein, the driving-rule 22 is distinguished from a traffic-law such as posted-speed limit as violating the driving-rule 22 does not imply that an infraction of traffic-laws has or will occur.

What constitutes the driving-rule 22 will become apparent in view of the several examples of the driving-rule 22 given below. It will also become apparent how strict adherence to the driving-rule 22 can sometimes result in reduced overall traffic efficiency, so in some situations it may be advantageous to violate or modify, possibly temporarily, the driving-rule 22 with the benefit being an increase in overall traffic efficiency. The decision to violate or modify the driving-rule 22 may be based on the detection of an observed-deviation 24 of the driving-rule 22 by the other-vehicle 14. That is, the modification of the driving-rule 22 may be based on the observed-deviation 24.

The operation of the host-vehicle 12 in accordance with the driving-rule 22 determines the behavior of the vehicle-operation 26 whether the host-vehicle 12 is being operated in an automated-mode, a manual-mode, or a partial blending of the automated-mode and the manual-mode. The driving-rule 22 is then influential as to how aggressively the vehicle-controls 28 are operated. The system 10 may also include a location-device 32 used to determine a location on a digital-map (not shown) so the system 10 can ‘remember’ locations where violation or modification of the driving-rule 22 occurs repeatedly.

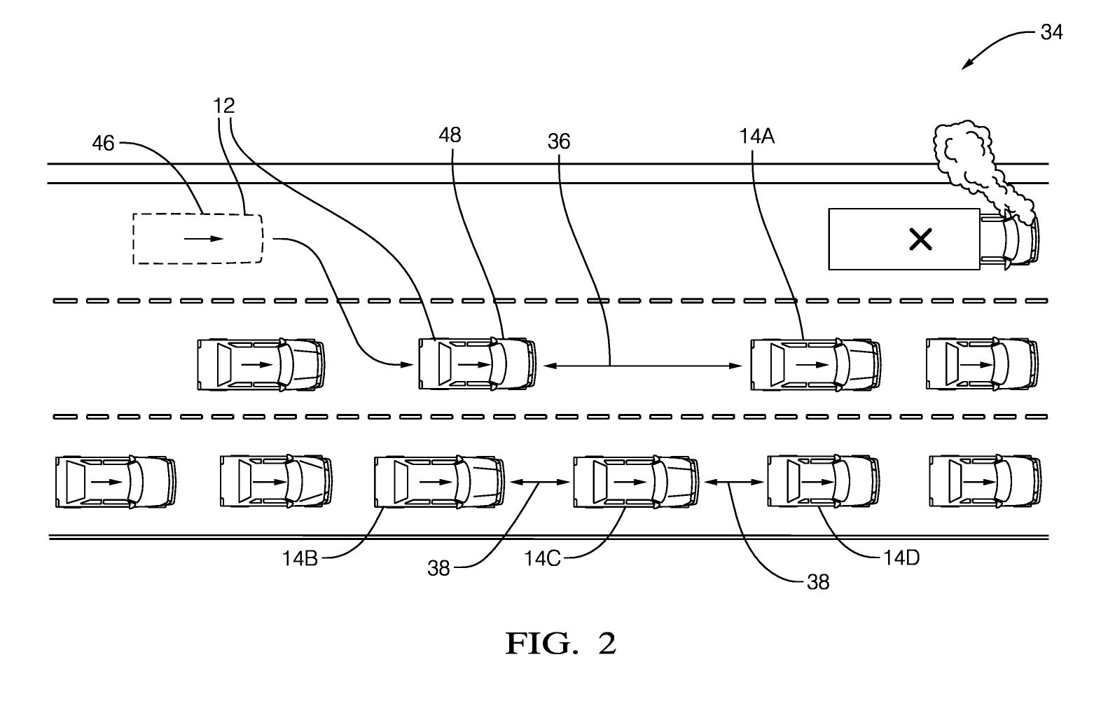

FIG. 2 illustrates a non-limiting example of a traffic-scenario 34. A non-limiting example of the driving-rule 22 may be a following-distance 36 that the host-vehicle 12 maintains or exceeds when following the other-vehicle 14A. The following-distance 36 may be a preprogrammed value stored in the controller 20. The observed-deviation 24 observed by the system 10 using the vehicle-detector 16 and/or the transceiver 18 may be a separation-distance 38 between other-vehicles 14B, 14C, 14D proximate to the host-vehicle 12 that is substantially different from the following-distance, e.g. less than 80% of the following-distance 36. If the system 10 observes that the typical value of the separation-distance 38 is substantially less than the stored value of the following-distance 36, the system 10 may modify the following-distance 36 based on the separation-distance 38. That is, the following-distance 36 may be decreased to, for example, an average of the observed value of the separation-distance 38 between the other-vehicles 14B, 14C, 14D. The modified value of the following-distance 36 may be temporarily or permanently maintained, or may only be used when the host-vehicle is traveling on the roadway illustrated in FIG. 2 as indicated by the location-device 32.

Continuing to refer to FIGS. 1 and 2, the host-vehicle 12 may encounter a stalled-truck 40 stalled in the left-lane of the roadway. A non-limiting example of the driving-rule 22 may be passing-only-on-left 42. That is, the system 10 may be pre-programmed to only allow the host-vehicle 12 to only pass any other vehicle on the left-hand side of the other-vehicle. However, if the observed-deviation 24 is a pas sing-on-right 44 by, the other-vehicles 14A, 14D for example, then the driving-rule 22 may be temporarily modified to allow passing-on-right by the host-vehicle 12. As illustrated, the host-vehicle may change-lanes from a prior-location 46 to a current-location 48 in preparation to pass the stalled-truck 40 on the right-hand side of the stalled-truck 40. As before, this modification may be a temporary modification so the driving-rule 22 is reset to the passing-only-on-left 42 after a few minutes.

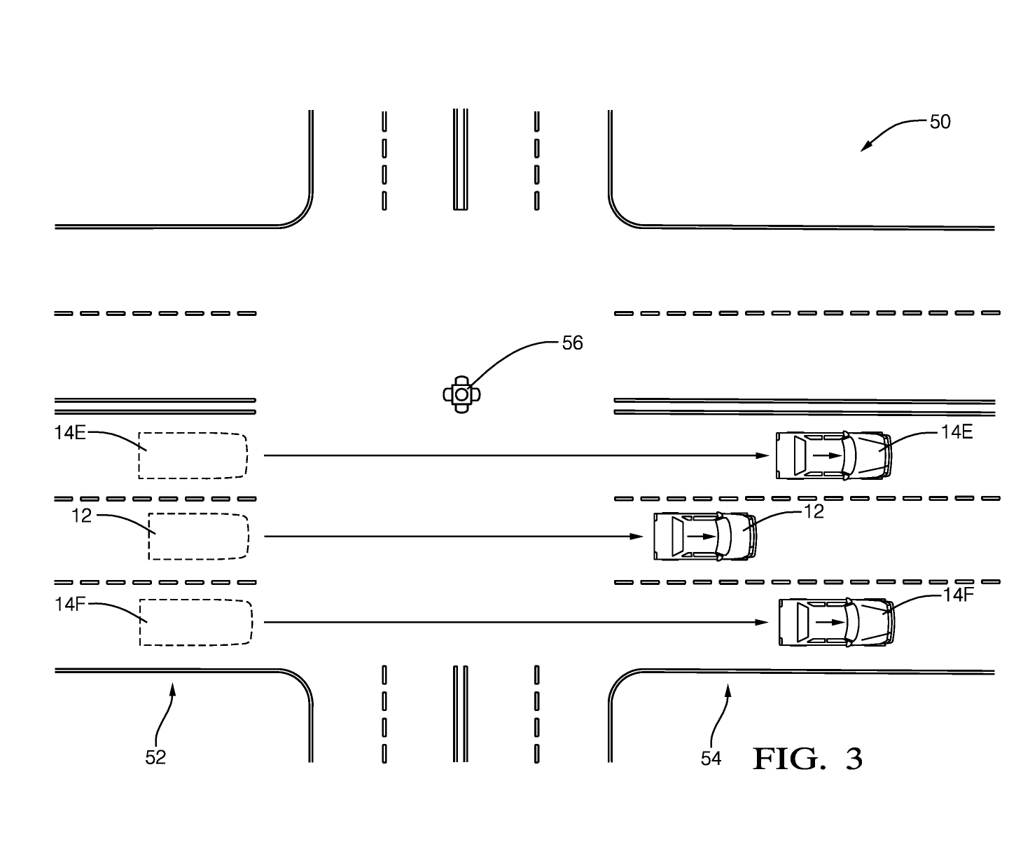

FIG. 3 illustrates another non-limiting example of a traffic-scenario 50 where the host-vehicle 12 and other-vehicles 14E, 14F are previously located at a prior-position 52 and are subsequently located at a current-position 54 after a traffic-signal 56 changes from red to green. The driving-rule 22 for the host-vehicle 12 may include an acceleration-rate 58 previously programmed into the controller 20. However, if the observed-deviation 24 is rapid-acceleration 60 by the other-vehicles 14E, 14F that is greater than the acceleration-rate 58 such that after reaching the current-position 54 the host-vehicle 12 is substantially behind the other-vehicles 14E and 14F, then the relatively slow acceleration by the host-vehicle 12 may unnecessarily impede following-vehicles (not shown) located behind the host-vehicle 12 and the other-vehicles 14E, 14F, possibly causing unnecessary lane-changes by the following-vehicles. Accordingly, the acceleration-rate 58 of the host-vehicle 12 may be increased to better correspond to the local driving habits exhibited by the other-vehicles 14E, 14F.

By way of further non-limiting examples, the driving-rule 22 may include a waiting-time (not shown) that the host-vehicle 12 waits at a yield-sign and/or a stop-sign before proceeding. The controller 20 may be configured to observe the waiting-time even if there are no other vehicles near the host-vehicle 12. For example, the controller 20 may be pre-programmed to wait for one second (1s) at a stop-sign before proceeding. However, if the other-vehicle 14 or other vehicles are observed (the observed-deviation 24) as waiting at stop signs for substantially more or less than the pre-programmed waiting-time, e.g. >1.5s or less than 0.5s, then the controller 20 may adjust the waiting time (the driving-rule 22).

By way of another non-limiting example, the driving-rule 22 may include a lateral-avoidance-distance (not shown) that the host-vehicle 12 maintains for clearance when passing a pedestrian or bicyclist (not shown) traveling on or near the edge of the roadway, two-meters (2m) for example. However, if the observed-deviation is that the other-vehicle 14 or other vehicles typically steer to provide three or more meters (>3m), then the controller 20 may modify the driving-rule 22 accordingly.

By way of another non-limiting example, the driving-rule 22 may include a steering-accuracy (not shown) that is a measure of how accurate the host-vehicle is steered to follow the center of the travel-lane. It has been observed that some human-operators tend to drive closer to the edge of the travel-lane when a roadway is curvy to allow for additional clearance for unseen until the last instant opposing traffic. If the controller 20 observes that the observed-deviation 24 is that most other vehicles are right-of-center rather than not centered, the controller 20 may adjust the driving-rule 22 accordingly.

Accordingly, a driving-rule system (the system 10), a controller 20 for the system 10 and a method of operating the system 10 is provided. While preprogramming of the driving-rule 22 may have been based on a desire for an exceptionally smooth or leisurely ride in the host-vehicle 12, local driving habits exhibited the other-vehicle 14 and/or the other-vehicles 14A, 14B, 14C, 14D, 14E, 14F may indicate that the driving-rule 22 be modified so the behavior of the host-vehicle 12 better corresponds with the local driving habits.

While this invention has been described in terms of the preferred embodiments thereof, it is not intended to be so limited, but rather only to the extent set forth in the claims that follow.

We claim:

1. A driving-rule system for an automated vehicle, said system comprising:
a vehicle-detector used to detect movement of an other-vehicle proximate to the host-vehicle; and
a controller in communication with the vehicle-detector, said controller configured to operate the host-vehicle in accordance with a driving-rule that allows passing-only-on-left, detect an observed-deviation of the driving-rule when the other-vehicle is observed passing-on-right, and then, in response to the other-vehicle passing-on-the-right, modify the driving-rule to allow passing-on-right by the host-vehicle.

2. The system in accordance with claim 1, wherein the driving-rule is reset to the passing-only-on-left one minute after the driving-rule is modified to allow passing-on-right by the host-vehicle.
